(19) United States
(12) Patent Application Publication
(10) Pub. No.: US 2010/0094156 A1
(43) Pub. Date: Apr. 15, 2010

(54) System and Method for Biofeedback Administration

(76) Inventor: Thomas F. Collura, Chagrin Falls, OH (US)

Correspondence Address: ROETZEL & ANDRESS, 1375 EAST 9TH STREET, CLEVELAND, OH, 44114, US

(21) Appl. No.: 12/250,361

(22) Filed: Oct. 13, 2008

(51) Int. Cl. A61B 5/04

(52) U.S. Cl. 600/545

(57) ABSTRACT

A biofeedback system for administration of electroencephalographic (EEG) neurofeedback training includes a plurality of electrodes sensors for placement on the head of a trainee and a switching head box comprising a plurality of contacts each of which connects to one electrode sensor and for specific biofeedback and neural connectivity training. The system also includes an interface device which includes at least two EEG signal amplifiers and connects to the switching head box, and a computer comprising software for generating user-control functions which corresponds in real-time to EEG signals received by the interface device and processed by the computer. The switching head box includes a switch with at least two conductors and connects the electrode sensors to the interface device for transmitting EEG signals from the trainee to the computer. Specific combinations of electrode sensors are used for specific types of biofeedback training.

### RELATED APPLICATION

This application is a continuation-in-part of U.S. patent application Ser. No. 11/512,949, filed Aug. 30, 2006.

### FIELD OF THE INVENTION

The invention pertains generally to EEG biofeedback for learning and controlling bio-electric characteristics of the brain which correspond to different mind states. More particularly, the invention relates to system and method for obtaining quantitative EEC measurements and values from sensors positioned at various locations of the brain.

### BACKGROUND

Biofeedback is the recording, monitoring and analyzing of electrical activity of the brain and a corresponding mental state of a user. A plurality of visual, auditory and/or tactile feedback mechanisms are (integrated) with the electrical activity of the brain to facilitate neurofeedback training of the user. The interface is provided in such a manner so as to provide the ability of the user, in the case of self-administered monitoring, or the trainer, in the case of an administered session, to record, manage and control brain activity for different purposes including self-improvement.

EEG (brainwave) signals have been extensively studied in an effort to determine relationships between frequencies of electrical activity or neural discharge patterns of the brain and corresponding mental, emotional or cognitive states. Biofeedback of identified frequency bands of EEG signals is used to enable a person to voluntarily reach or maintain a target mental state. Frequency bands of EEG readings used in such biofeedback have been generally categorized in the approximate frequency ranges of: delta waves, 0 to 4 Hz; theta waves, 4 to 7 Hz; alpha waves, 8 to 12 Hz; beta waves, 12 Hz to 36 Hz, and sensorimotor rhythm (SMR) waves, 12 to 15 Hz.

It is theorized that each of the major subbands of biofeedback EEG (delta, theta, alpha, and beta) has unique bio-electric characteristics which correspond with unique subjective characteristics of an individual. The delta band is observed most clearly in coma and deep sleep, the theta band in light sleep and drowsiness, the alpha band in a variety of wakeful states involving creativity, calm and inner awareness, and the beta band in alert wakeful situations with external focus. In general, a dominant brain wave frequency increases with increasing mental activity.

Many different approaches have been taken to EEG biofeedback to achieve mental state control. For example, U.S. Pat. No. 4,928,704 describes a biofeedback method and system for training a person to develop useful degrees of voluntary control of EEG activity. EEG sensors are attached to cortical sites on the head for sensing BEG signals in a controlled environment. The signals are amplified and filtered in accordance with strict criteria for processing within time constraints matching natural neurologic activity. The signals are filtered in the pre-defined subbands of alpha, theta, beta and delta, and fed back to the monitored person in the form of optical, aural or tactile stimuli.

QEEG devices typically record a minimum of 19-20 channels, for data acquisition and analysis to map brain activity. These devices have individual EEG signal amplifiers for each channel and are expensive and complicated systems to run, requiring an expert in the field to conduct training. Currently, substantially less expensive systems which have a lower number of channels, for example, two to four channel devices, which include an amplifier for each channel, can also be used. However, in a two-channel interface device, for example, the trainee or trainer is required to take additional time to reposition the conductors to two different sites on the head for each recording. Thus, in many of the conventional EEG biofeedback systems and methods, it is necessary to interrupt data collection to reposition the conductors, and in some cases, to also perform set-up functions, review component values, or set protocols or adjust threshold levels. These functions are typically performed by a session administrator, which can ultimately diminish or otherwise adversely affect the nature and quality of biofeedback signals to a trainee seeking to benefit from EEG training.

### SUMMARY

The present invention provides for a system, program and method of recording brainwaves around the head quickly and cost effectively on a low number of channels relative to a QEEG system. It provides recording from a relatively low number of channels to multiple sensor locations, and also provides a system and method to switch between channels instantly to obtain quality biofeedback.

In one embodiment, the present invention provides for a system for administration of electroencephalographic (EEG) neurofeedback training which includes a plurality of electrode sensors for placement on the head of a trainee, a switching head box electrically connected to the at least two sensors, an interface device which includes at least two EEG signal amplifiers and is electrically connected to the switching head box, and a computer electrically connected to the interface device and which includes software for generating user-control functions which correspond in real time to EEG signals received by the interface device. The switching head box includes a switch having a first conductor at a first position which connects a first electrode sensor to a first EEG signal amplifier of the interface device, and a second conductor at a second position which connects a second electrode sensor to a second EEG signal amplifier, for transmitting EEG signals from the trainee to the computer.

In another embodiment of the invention, a program embodied in a computer readable medium includes logic that simultaneously identifies at least two independent BEG brainwave signals received by at least two electrical sensors placed on a head of a trainee undergoing biofeedback training. The program includes logic which executes processing of the EEG brainwave signals and records EEG brainwave data derived from the EEG brainwave signals and logic that detects a predetermined time setting for processing the EEG brainwave signals and executes a prompt, at the conclusion of the predetermined time setting, to advance a switch if additional electrical sensors are to be processed.

### BRIEF DESCRIPTION OF THE SEVERAL VIEWS OF THE DRAWINGS

The various embodiments of the present invention can be understood with reference to the following drawings. The components in the drawings are not necessarily to scale. Also, in the drawings, like reference numerals designate corresponding parts throughout the several views.

FIG. 1 is a block diagram of the hardware components of a biofeedback system according to an embodiment of the invention;

FIG. 2 is a schematic diagram of the biofeedback system of FIG. 1, according to an embodiment of the invention;

FIG. 3 is an electrical schematic diagram of a two-channel, six-position switching head box of the biofeedback system of FIG. 1, according to an embodiment of the invention;

FIG. 4 is an electrical schematic diagram of a four-channel, 5-position switching head box of the biofeedback system of FIG. 1, according to an embodiment of the invention;

FIG. 5 is an electrical schematic diagram of a 2-channel, 2-position switching head box of the biofeedback system of FIG. 1, according to an embodiment of the invention;

FIG. 6 is a flow chart that provides an example of the logic that is executed in the controller of an interface device of the biofeedback system of FIG. 1, according to an embodiment of the invention; and

FIG. 7 is a screen display generated by monitoring logic of the biofeedback system of FIGS. 1 and 2, according to an embodiment of the invention.

FIG. 8 is a diagram of the location of electrode sensors for biofeedback system position 1.

FIG. 9 is a diagram of the location of electrode sensors for biofeedback system position 2.

FIG. 10 is a diagram of the location of electrode sensors for biofeedback system position 3.

FIG. 11 is a diagram of the location of electrode sensors for biofeedback system position 4.

FIG. 12 is a diagram of the location of electrode sensors for biofeedback system position 5.

FIG. 13 is a diagram of the location of electrode sensors for biofeedback system position 5a.

FIG. 14 is a diagram of the location of electrode sensors for biofeedback system position 6.

FIG. 15 is a diagram of the location of electrode sensors for biofeedback system position 7.

FIG. 16 is a diagram of the location of electrode sensors for biofeedback system position 8.

FIG. 17 is a diagram of the location of electrode censor for biofeedback system position 9.

### DESCRIPTION OF EMBODIMENTS

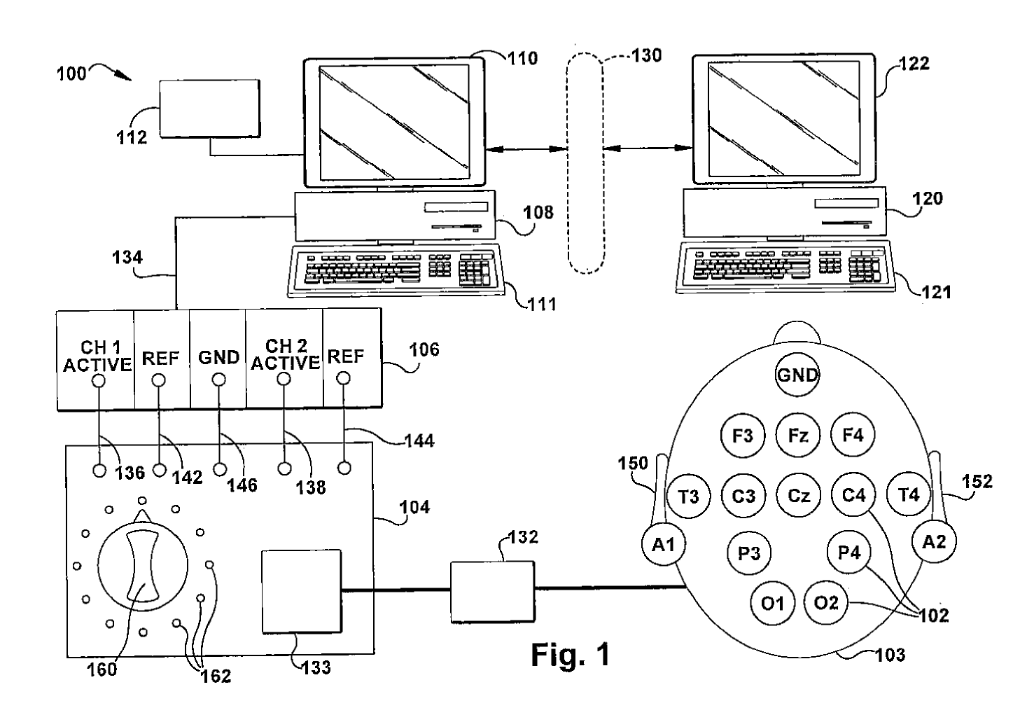

FIG. 1 is a block diagram of the hardware components of a biofeedback system 100 according to an embodiment of the invention. The biofeedback system 100 includes a plurality of electrodes 102 attachable to an electro-cap that is placed on the head 103 of a subject or trainee undergoing biofeedback training. The biofeedback system 100 further includes a switching head box 104, a user interface device 106, and a trainee computer or data processor 108 which is electrically connected to a display monitor 110, keyboard 111, and optionally, additional biofeedback stimulative devices 112 such as audio or vibratory headphones, light goggles, and/or tactile stimulator. These devices may be controlled by a feedback device controller (not shown) connected to user computer 108. The user computer 108 contains BEG analysis and biofeedback software which performs EEG recording, analysis and biofeedback operations, as will be further described herein. The biofeedback system can optionally include a trainer computer 120 having keyboard 121 and display monitor 122, in which the trainer computer 120 is connected to the trainee computer 108 either as another computer in a networked environment or at a remote location via the internet 130.

The EEG signals from the trainee undergoing biofeedback training flow from electrodes which connect to the switching head box 104 via a pigtail connector 132 or individually to individual pin-type connections (not shown) to connector 133 on the switching head box 104. The interface device 106 electrically connects to the trainee computer 108 via cable connector 134 and interface device 106 electrically connects to the switching head box 104 through various serial data lines, for example line 136 to channel 1 (CH 1), line 138 to channel 2 (CH 2), lines 142 and 144 to reference and line 146 to ground. The switching head box 104 includes a selector switch 160 that can be turned to a plurality of positions 162. The selector switch 160 allows the trainee or trainer to easily select the electrodes for data collection and to control the reading of various areas of the head that are transmitting BEG data to the trainee computer 108. Thus the selector switch 160 prevents the trainee or trainer from having to move the electrodes to various positions on the head in order to obtain several EEG readings. The trainee can use a standard EEG cap and can easily select various areas of the brain in a short time. Furthermore, the software within the trainee computer 108 can prompt the trainee or trainer to switch the channels at a pre-determined time period to collect data at several electrodes to complete a biofeedback training session, as will be further discussed. Therefore, switching head box 104 allows the trainee or trainer to select which electrodes will be transmitted through to the interface device 106 and sent to the trainee computer to be read by the software therein. The interface device 106 reads the EEG signals coming into lines 136 and 138 and converts them to digital form, and sends the digital signals to the computer 108 and the signals can then be viewed and interpreted on software, for example, Windows Operating System.

FIG. 1 also shows location of the plurality of electrodes 102 attached to the trainee head 103 as, for example a neutral (or “indifferent”) electrode to each ear 150, 152, electrodes A1 and A2, and at least one electrode to locations on the scalp, for example, one on each side of the forehead C3 and C4 to provide “right active” and “left active” two-channel input, and a “ground” GND electrode. Generally, the active electrode will be attached to the head in a specific location (frontal, parietal, occipital, etc.), and the indifferent and ground electrodes will be attached to each ear 150, 152. The active and indifferent electrodes connect through the switching box 104 and then to the interface device 106. For example, when the selector switch 160 is turned to a single position of the plurality of switch positions 162, and with the active electrodes C3 and C4 attached to the head 103, the indifferent electrodes A1 and M attached to the left 150 and right ears 152, the switching head box 104 and the interface device will track (measure) brainwave activity between the head and the left and right ears as references, and sensor GND on forehead used as ground. Therefore, in one example embodiment, two active leads C3 and C4 can provide EEG monitoring through channel 1, CH 1, and channel 2, CH 2, respectively, of the interface device 106.

In addition, several additional active leads may connect to channels 1 and 2, respectively. For example, when the selector switch 160 is turned to a single position, of the plurality of switch positions 162, active electrodes C3, C4 can provide monitoring through channel 1 and electrodes P3 and P4 can provide monitoring to channel 2. Selector switch 160 may then be turned to a new position and active electrodes T3, T4 can provide monitoring through channel 1 and electrodes O1, O2 can provide signals through channel 2. Therefore two or more electrode connections can be read in channel 1 while two or more electrode connections can be read in channel 2. The selector switch 160 can then be turned so that additional electrodes may be read via channels 1 and 2. In an alternative embodiment, the switching head box 104 can have additional channels, for example 10 or more channels.

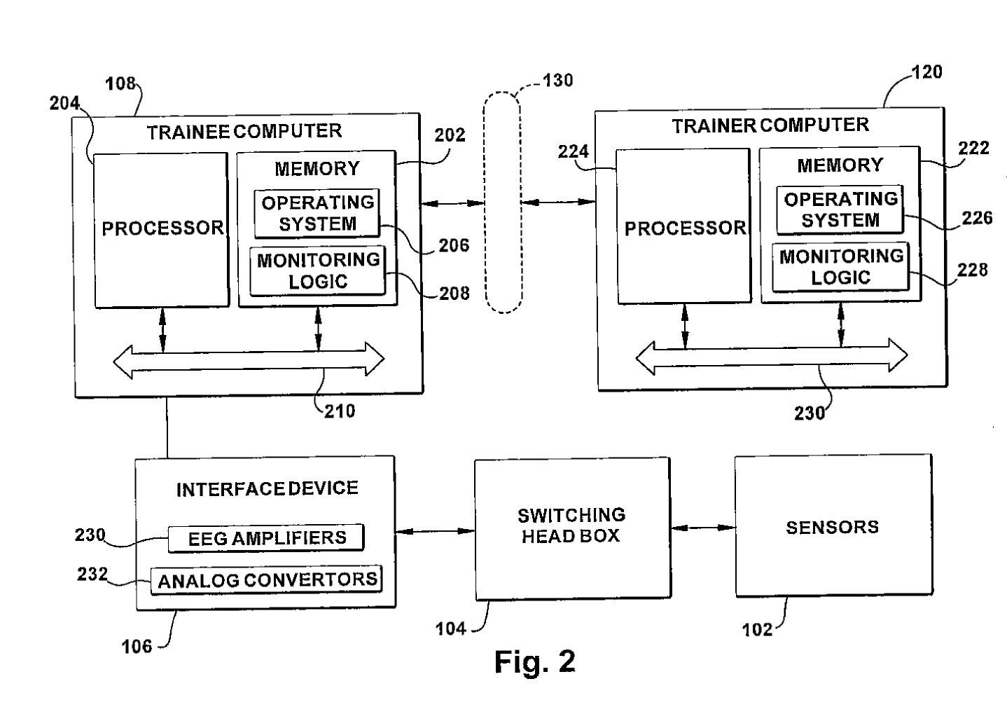

FIG. 2 is a schematic diagram of the biofeedback system of FIG. 1 which includes the sensors 102, switching head box 104, interface device 106, trainee and trainer computers 108, 120 all of which are electrically coupled to one another. The example embodiment of FIG. 2 is described with reference to a trainee computer 108 that is directly coupled to interface device 106 which selectively reads EEG signals via sensors 102 on trainee head through switching head box 104. The trainee computer 108 could be directly coupled to trainer computer 120, or alternatively, the trainee computer 108 could interface with a trainer computer 120 in a networked environment or via the Internet, intranets, wide area networks (WANs), local area networks, wireless networks, or other suitable networks, etc., or any combination of two or more such networks. The trainee and trainer computers 108, 120 may be, for example, desktops, laptops, palm or hand held computers such as a personal digital assistant, or any other devices with like capability.

The trainee computer 108 includes software or firmware components that are stored in the memory 202 and are executed by the processor 204, and each are coupled to respective local interface 210, for example an input/output data bus which can also connect to keyboard 111 and biofeedback stimulative devices 112 (FIG. 1). The trainer computer 120, if present, also includes software or firmware components that are stored in the memory 222 and are executable by the processor 224, and are coupled to local interface 230. These components include, for example, operating systems 206, 226 and monitoring logic 208, 228. The operating systems 206, 226 are executed to control the allocation and usage of hardware resources such as the memory, processing time and peripheral devices 111, 112, 121 (FIG. 1). In this manner, the operating systems 206, 226 serve as the foundation on which applications depend. Monitoring logic 208, 228 monitors trainee EEG signals and provides feedback for biofeedback training. For example, the monitoring logic 208 of trainee computer 108 may include logic that performs EEG signal processing for EEC frequency band measurement and to generate images of these brainwave measurements, logic that makes a determination of the information via computation functions, logic that carries out a number of possible user feedback tasks which can be displayed on trainee monitor 110 (FIG. 1), logic that sorts, saves and restores data files, and logic which provides summary reporting and graphing capabilities.

As used herein, the term “executable” means a program file that is in a form that can ultimately be run by the processors 204, 224. Examples of executable programs may be, for example, a compiled program that can be translated into machine code in a format that can be loaded into a random access portion of the memories 202, 222 and run by the processors 204, 224 or source code that may be expressed in proper format such as object code that is capable of being loaded into a random access portion of the memories 202, 222 and executed by the processors 204, 224 etc. An executable program may be stored in any portion or component of the memories 202, 222 including, for example, random access memory, read-only memory, a hard drive, compact disk (CD), floppy disk, or other memory components.

The memories 202, 222 are each defined herein as both volatile and nonvolatile memory and data storage components. Also, each of the processors 204, 224 may represent multiple processors and each of the memories 202, 222 may represent multiple memories that operate in parallel processing circuits, respectively. In such a case, each of the local interfaces 210, 230 may be an appropriate network that facilitates communication between any two of the multiple processors, between any processor and any of the memories, or between any two of the memories, etc.

The interface device 106 acquires and transmits data, and the trainee computer 108 receives and processes the data to make a determination of the information, and then carries out any of a number of possible user-feedback tasks which can be displayed the display monitor 110 (FIG. 1) connected to the user computer 108. As mentioned above, interface device 106 receives data from sensors 102 via switching head box 104 through the data serial lines 136, 138 (FIG. 1) which it transmits to the trainee computer 108. Interface device 106 includes two or more BEG signal amplifiers 230, one for each channel of data transmission. As shown in FIG. 1, the interface device 106 is transmitting 2 channels of data and therefore has two EEG signal amplifiers 230, although additional channels of data are possible, for example 2-10 channels, in another example, 2-8 channels, 2-6 channels, 2-4 channels and all combination of numbers of channels there between. Interface device 106 includes firmware in the way of analog converters 232 which read the incoming analog EEG signals from electrode sensors 102, converts them to digital form, and sends the digital signals to the trainee computer 108. The digital signals can then be viewed and interpreted on software installed on the trainee computer 108, as will be further described.

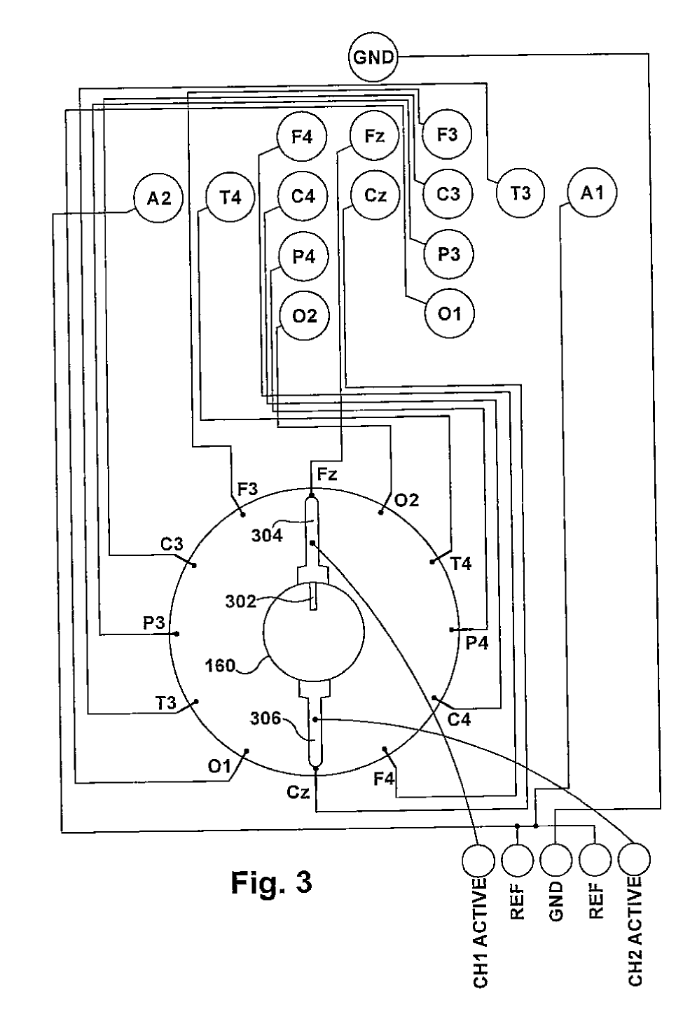

Next, a general description of the operation and functioning of switching head box 104 is provided within the context of the biofeedback system 100 of FIGS. 1 and 2. FIGS. 3 through 5 show example electrical schematics of switching head box 104 configured to receive data from the electrode sensors 102 (FIGS. 1 and 2) and to transmit the data to the interface device 106 (FIGS. 1 and 2) through two or more channels. Each electrical schematic illustrates one of several possible electrical circuits are established via the switch 160 at the various switch positions that may be selected. As illustrated, the switch 160 has two conductors 304, 306 which make contact with the electrode sensors 102 and two active channel ports, CH1, CH2. Each conductor 304, 306 connects to one electrode sensor 102, and so, switch 160, as shown, connects to two electrode sensors on the head of the trainee undergoing biofeedback treatment when the switch is located at each switch position 160. As stated above and as shown in the example embodiments described below, each channel of the switching head box 104 interfaces with a separate amplifier of the interface device 106 through channel ports CH1, CH2.

In the example embodiment shown in FIG. 3, data is transmitted from a first electrode sensor FZ located on the head of the trainee undergoing feedback treatment to contact FZ of switching head box 104, and through conductor 304 of switch 160 which electrically connects to Channel 1 port, CH1, to interface device 106. At the same time, data from a second electrode sensor, CZ located on the head of the trainee is transmitted through conductor 306 of switch 160 and to Channel port two, CH2 of switching head box 104 to interface device 104. After the data is passed through separate EEG signal amplifiers of interface device 106, the data is transmitted to trainee computer 108 (FIG. 1). Therefore, activation of switch 160 to a first position as indicated by position indicator 302 completes two electrical circuits that allows current to pass through two separate electrode sensor sites of the brain to the interface device 106 and to the trainee computer 108. In the embodiment shown, the switch 160 can be turned to six positions in which the conductors make contact with all twelve electrode sensor sites. A suitable switch can be any switch, for example, a double-pole switch that can move to two or more positions and that is capable of completing at least two electrical circuits that connect two electrode sensors to two distinct channel ports, CH 1 and CH 2, of switching head box 104 and to interface device 106 and to two distinct EEG signal amplifiers, of interface device 104. The switching head box 104 of FIG. 3 is designed such that switch 160 has two conductors that interface with 12 electrode sites and where the switch 160 can be moved to six positions to read data to electrode sensor sites at each switch position. Accordingly, when switch 160 is placed in a second, third, fourth, fifth and sixth electrical contacts at six positions, electrical contact is made and therefore data can be read from electrode sensor pairs C3 and C4, P3 and P4, T3 and T4 and O1 and O2, respectively.

In alternative embodiments, switching head box 106 can be configured to receive data from a large range of electrode sensor sites. For example, the number of sensor sites that can be read depend on the number of electrode sites or the electrode cap that is placed on the head of the trainee and can range anywhere from 2-256 sites and another example can range from 2-64, and another embodiment from about 2-32 and in another embodiment from about 2-20, and in still yet in another embodiment from about 2-12 electrodes and all ranges there between. In addition, switch 160 of switching head box 104 can include at least two conductors, depending upon the number of channel ports and channels that can be read by interface device 106.

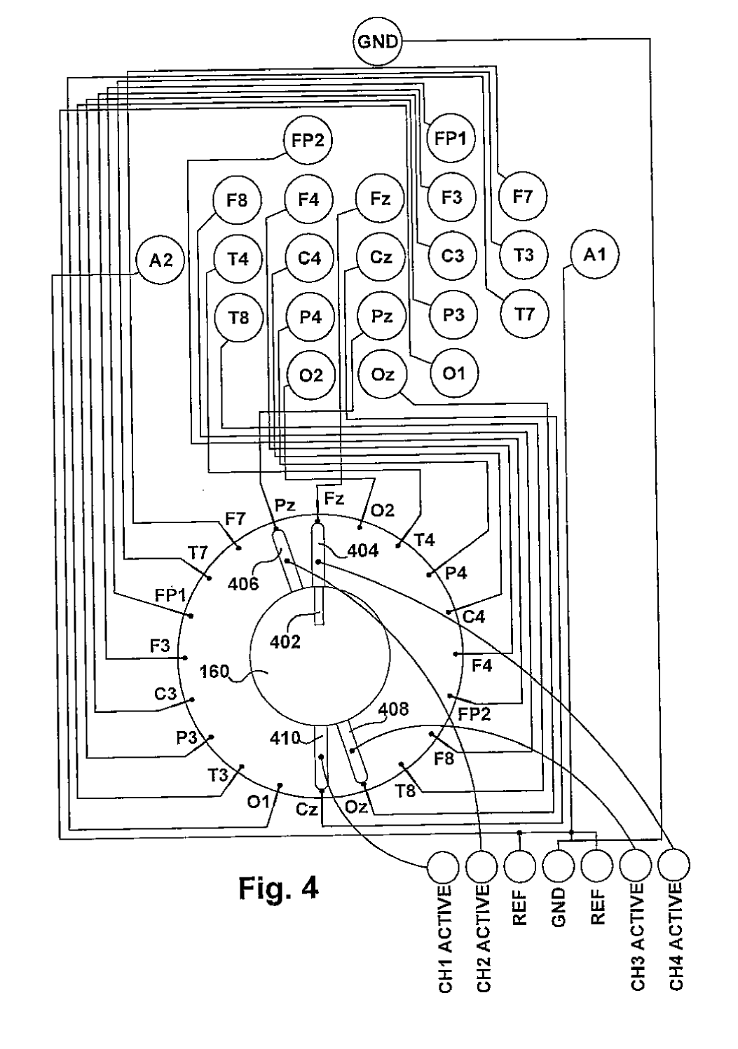

FIG. 4 illustrates an electrical schematic of a switching head box 104 that reads data from 20 electrode sensor sites. In addition, switching head box 106 includes four distinct channel ports, CH1, CH2, CH3 and CH4, which can allow for the transmission of data for four separate EEG signal amplifiers of interface device 106. Switch 160 has four conductors, 404, 406, 408, 410, which make contact with four contacts at four positions to read four distinct electrode sensors. Switch 160 can be rotated to five different positions in order to transmit the data from all twenty electrode sensor sites. Accordingly, switching head box 104 of FIG. 4 is a four channel, 5 position switching head box 104. When switch 160 is placed in a first position, as indicated by position indicator 402, contact 404 makes contact with electrode site FZ, contact 406 makes contact with electrode sensor site PZ, electrode 408 makes contact with electrode sensor site OZ and conductor 410 makes contact with electrode sensor site CZ. As shown, data from electrode sensor site CZ is transmitted to the Channel 1 port, CH1, the data from electrode sensor site PZ is transmitted to Channel port 2, CH 2, the data from electrode sensor site OZ is transmitted to Channel 3 port, CH 3, and the data from electrode sensor site FZ is transmitted to Channel port 4, CH4. Thus, four separate circuits can be established simultaneously through switch 160 of switching head box 106. Movement of switch 160 to a second position breaks the circuit to electrode sites Cz, Pz, Oz and Fz and establishes connection to four new sites, for example, T4, P4, P3, and T3. Since, in the embodiment of FIG. 4, the number of electrode sites is 20, the switch can be placed in a third, fourth and fifth position to make electrical contact with electrode sites P4, C4, P3 and C3; and F4, FP, 2, F3, and FP1; and F8, T8, T7 and F7, respectively.

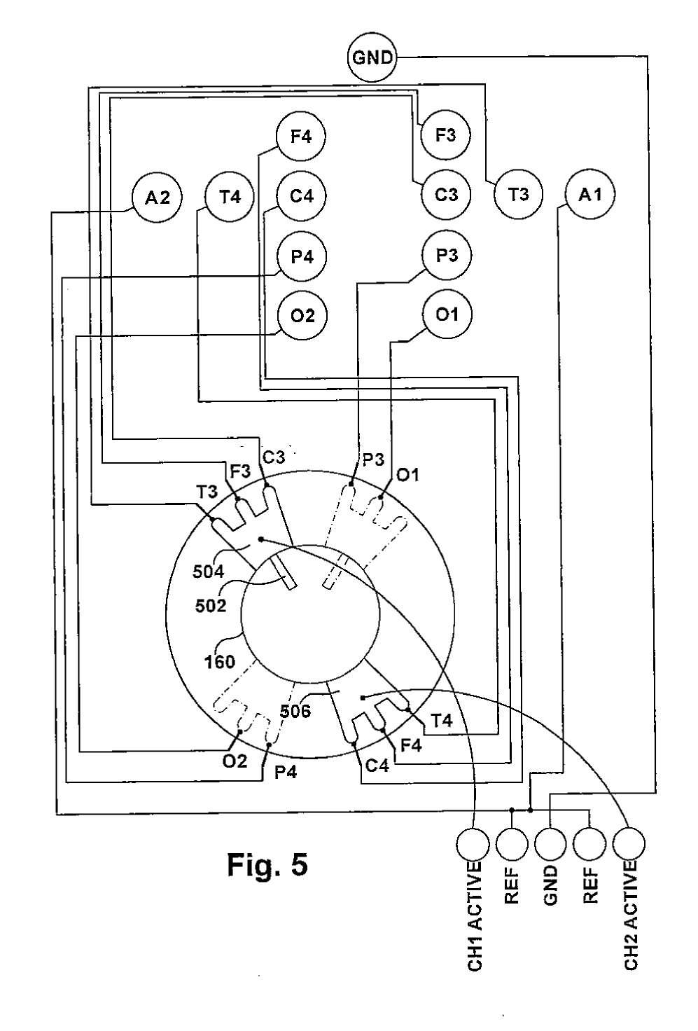

It should be understood, that any four sensors can be chosen for connection at a given time. For example, although electrode sensors Fz, Pz, Oz are shown to make connection at the same time, other alternative sites can be made by conductors 404, 406, 408 and 410. Thus, in the example embodiments of FIGS. 4 and 5, each conductor makes contact with one electrode sensor site and transmits data to a single EEG signal amplifier. In addition, it is also possible that switch 160 of FIG. 4 includes two conductors, for example, or any number of conductors greater than two.

In conducting biofeedback training, it may be desirable to train whole sections of the brain. The biofeedback system 100 can also conduct training based on combined signals to perform a computation of coherence which is known as “synchrony training”. FIG. 5 shows a switch 160 having at least two conductors which receives data from at least two electrode sensor sites, at each electrode. For example, switch 160 is at a first position as indicated by indicator position 502, and contact is made to electrode sensors F3, T3 and C3 which are connected in parallel to provide a first channel reading to channel 1 port, CH1. Contact is also made via contact 506 to electrode sensors F4, T4 and C4 which are connected in parallel to provide a second channel reading which is transmitted to channel 2 port, CH 2. Therefore, each of the conductors 504 and 506 of switch 160 make connection to more than one sensor which transmits to each channel, and so data from the several electrode sensors are provided with only two EEG signal amplifiers. This electrical arrangement in conjunction with computation performed by the logic provides an average reading of the electrical activity of at least two electrode sensor sites. This method may be referred to as “volume-conduction averaging” and is a method for training multiple brain sites. This allows for sychrony training that is sensitive to the amplitude and phase synchrony of the different sites. Switch 160 can then be moved clockwise so that the position indicator 502 aligns with the second position and conductor 504 makes contact with electrode sensors P3, O1 and conductor 506 makes contact with P4 and O2, which transmits signals to Channel 1 port, CH1 and Channel 2 port, CH2, respectively.

The specific combination of sensors is a matter of design choice and can be variable. That is, the specific numbers or pairs or quads, etc., and combinations of sensors employed depend upon the desired training. Homologous pairs can be chosen such that contact 504 connects to all sensors on the left side of the brain, for example electrode sensors F3, T3, C3, P3, O1, and conductor 506 connects all sensors on the right side of the brain, for example, electrode sensor sites F4, T4, C4, P4, O2. Therefore synchrony training can conduct the entire head training with 10 sites being read through CH1 and 10 sites being read through CH2. Again, it should be understood that the number of electrode sensors read can vary greatly and the number of conductors of switch 160 can be any number greater than two, each of which connects to a distinct channel amplifier of interface device 106.

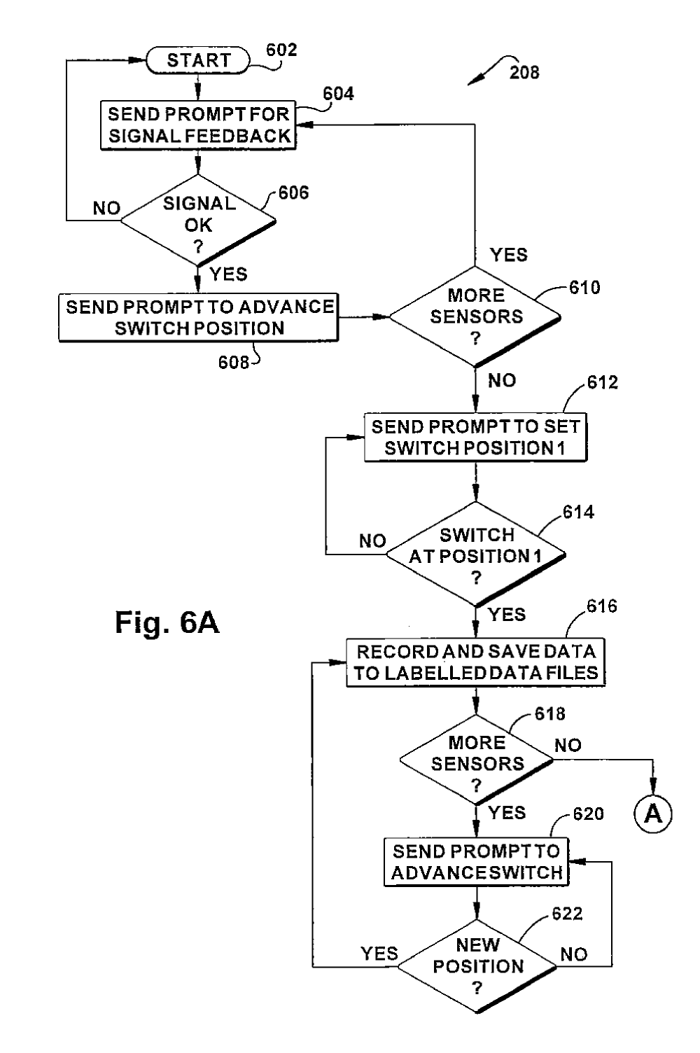

FIGS. 6A and 6B is a flow chart that provides an example embodiment of the monitoring logic 208 (FIG. 2) that is executed in a trainee computer, and optionally, a trainer computer of the biofeedback system of FIG. 1, according to an embodiment of the invention. FIGS. 6A and 6B show a flow chart of one example of the monitoring logic 208 according to an embodiment of the present invention. Alternatively, FIGS. 6A and 6B may be viewed as depicting steps of an example of a method implemented in a trainee computer 108 (FIG. 2) to determine the biofeedback readings of several sensors on the trainee's head. The functionality of the monitoring logic 208 as depicted by the example flow chart of FIG. 6A and B may be implemented, for example, in an object-oriented design or in some other suitable programming architecture. Assuming the functionality is implemented in an object-oriented design, each block represents functionality that may be implemented in one or more methods that are encapsulated in one or more objects. The monitoring logic may be implemented using any one of a number of programming languages such as, for example, C, C++, JAVA, Perl, or other suitable programming languages.

Beginning with box 602, the monitoring logic 208 sends a prompt at box 604 to the user, for example via display monitor 110 of computer 108, and the logic at box 606 determines whether or not the signal is sufficiently strong. Assuming that the signal is good, then at box 608 a prompt is sent to advance the switch position. The monitoring logic 208 then determines at box 610 whether or not there are any more signals from electrode sensors to be read for data. If the response is “Yes” then another prompt is sent for signal feedback at box 604 and to determine whether the signals from additional electrode sensors are sufficiently strong at box 606. If all of the signals are not sufficiently strong, then the monitoring logic starts over at 602.

Once there are no more electrode sensors to be read, then in box 612 the monitoring logic 208 sends a signal to prompt the user to set the switch position to the first switch position. The monitoring logic 208 then determines whether or not the switch has been advanced to the first position in box 614. If the switch position has not been set to position 1, the prompt will continue to be sent to the monitor 110 of the trainee computer 108. Once the switch position is set to position 1, the monitoring logic then records and saves data at box 616 to labeled data files within the memory 202 of trainee computer 108. Once that data is recorded and saved, the monitoring logic 208 determines whether there are any additional sensors to be read at box 618.

Assuming there are more sensors to be read, then the monitoring logic 208 sends a prompt to advance the switch at box 620. The monitoring logic then determines, at box 622, whether or not the switch has been advanced to a second position. Once the switch has been set to a second position, then the monitoring logic 208 records and saves the data to the labeled data files at box 616. This process starting at box 616 is repeated until all of the sensors have been read and the data have been saved and labeled to the data files. Once all of the data from all of the sensors have been read, then at box 624 the monitoring logic executes calculations and interpretations on the data. Once all the calculations have been executed, then the monitoring logic closes the data files at box 626 and then a prompt is sent to the user to identify images at box 628.

Next, the user can determine whether or not he or she wants to view the data that is being stored and labeled at box 630 where a prompt is sent to request action on the part of the user as to whether or not they want to view the data. If there is no interest in viewing the data, then the user can indicate “No” and the program will end. However, if the trainee and user wishes to view the data, then monitoring logic 208 sends a display menu at box 634, for example to the monitor 110 of the trainee computer 108. The logic then asks whether or not a particular image to be viewed has been identified by the user or trainee at box 636. If a choice of image has not been identified, then the monitoring logic will maintain the display prompt. However, once the trainee or user indicates a choice of the image to be identified from the display menu, then the monitoring logic at box 638 will display the data. Once the data has been displayed the monitoring logic provides the choice as to whether or not the trainee or user would like to see additional views of the data at box 640. Once the user has responded to the prompt “Yes” to see additional display menus, then the logic determines whether another image has been identified from the display menu in response to the prompt. Once a response to the prompt has been made by the user or trainee, then additional data can be displayed. The monitoring logic 208 will continue to prompt the user until the user responds to the prompt with a “No”, in which case the program will end at 642.

Thus, in one example embodiment of the invention, the monitoring logic 208 is configured such that it will continue to read all of the sensors and once the sensors have been read, prompts will be sent to change the switch position until the user or trainee no longer advances the switch positions. If the user responds that there are no more sensors to be read, then the monitoring logic continues into the calculation mode and display mode, in which case the user has several choices by which it can view images of the data and the calculations performed on the data.

Although the flow chart of FIGS. 6A and B shows a specific order of execution, it is understood that the order of execution may differ from that which is depicted. For example, the order of execution of two or more blocks may be changed relative to the order shown. Also two or more blocks shown in succession in FIGS. 6A and B may be executed concurrently or with partial concurrence. In addition, any member of counters, state variables, warning semaphores, or messages might be added to the logical flow described here, for purposes of enhanced utility, accounting, performance measurement, or providing trouble shooting aids, etc. It is understood that all such variations are within the scope of the present invention.

Although the monitoring logic 208 is embodied in software or code executed by general purpose hardware as discussed above, as an alternative each may also be embodied in dedicated hardware or a combination of software/general purpose hardware and dedicated hardware. If embodied in dedicated hardware, the monitoring logic 208 can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, programmable gate arrays (PGA), field programmable gate arrays (FPGA), or other components, etc. Such technologies are generally well known by those skilled in the art and, consequently, are not described in detail herein.

Also, where the monitoring logic 208 comprise software or code, each can be embodied in any computer-readable medium for use by or in connection with an instruction execution system such as, for example, a processor in a computer system or other system. In the context of the present invention, a “computer-readable medium” can be any medium that can contain, store, or maintain the monitoring logic 208 for use by or in connection with the instruction execution system. The computer readable medium can comprise any one of many physical media such as, for example, electronic, magnetic, optical, electromagnetic, infrared, or semiconductor media. More specific examples of a suitable computer-readable medium would include, but are not limited to, magnetic tapes, magnetic floppy diskettes, magnetic hard drives, or compact discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

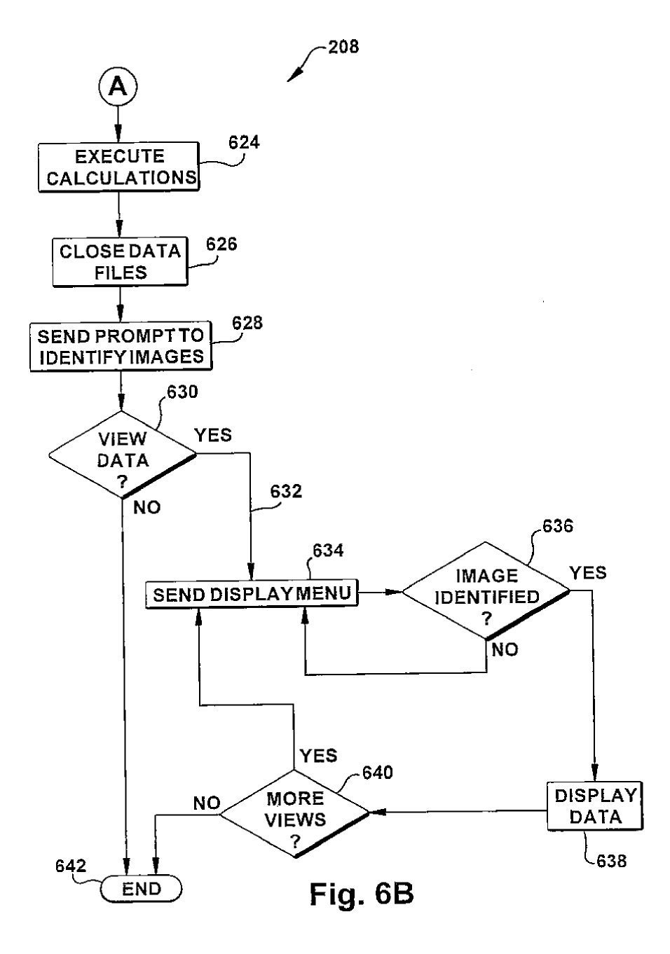

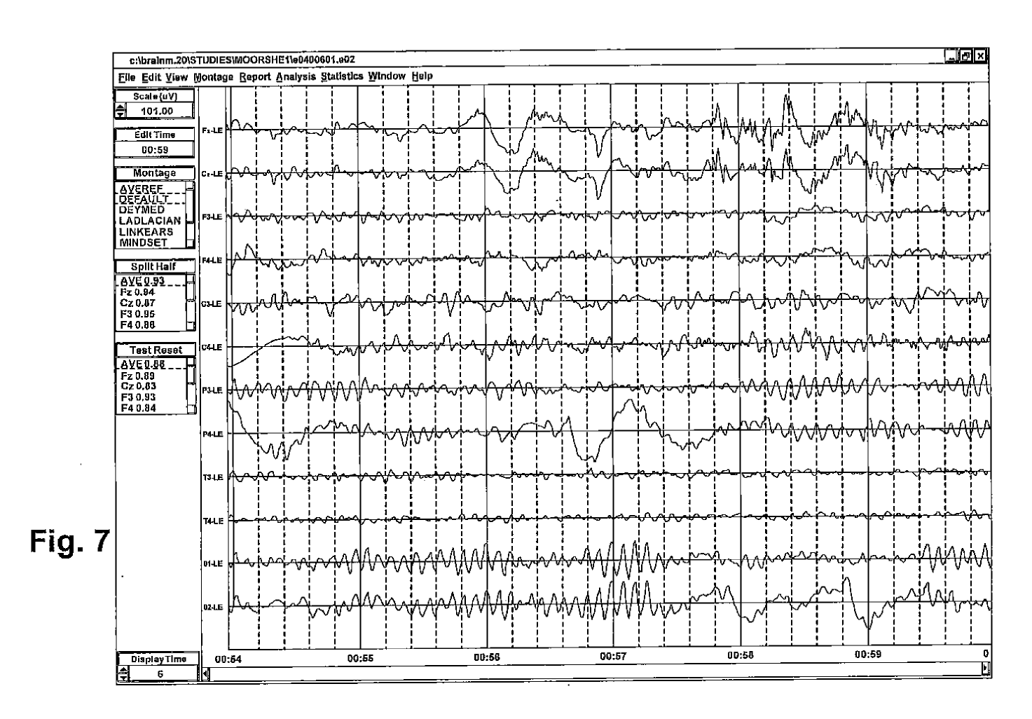

FIG. 7 illustrates an example of an EEG wave form signal display of a scrolling raw wave form using one configuration of the biofeedback system of the present invention. The wave form displays a test protocol, for example, which records a series of six, one second epochs of therefore displaying one second of EEG monitoring at each of (how many? 12?) electrode sensors at (six?) different switch positions. The data can be obtained without disturbing the neurofeedback training session. A trainee can use a standard EEG electrocap having a plurality of electrode sensor positions. Also, the length of time can vary at each electrode sensor position, for example to one minute intervals for each of the six positions, thereby completing the analysis in six minutes. This capability allows for the application of self-administered biofeedback training which eliminates the need for a dedicated operator or session administrator to monitor waveforms, independent of the trainee's activity.

Table I displays the EEG data derived from the EEG signals, for example, a textual summary of the EEG component values, their means, and standard deviations, for predetermined time intervals, or whenever prompt to a response is made.

TABLE I

| | | | | | | | | | | | | TH/ | | TH/ | |
|---|---|---|---|---|---|---|---|---|---|---|---|---|---|---|---|
| RUN | NPTS | SITE | TYPE | DELTA | THETA | ALPHA | LOBET | BETA | HIBET | GAMMA | USER | AL | TH/LB | BE | AL/BE |
| 1 | 60 | Fz | MEAN | 10.46 | 8.38 | 9.51 | 6.31 | 12.08 | 9.95 | 15.19 | 4.71 | 0.88 | 1.33 | 0.69 | 0.79 |
| 1 | 60 | Fz | MEANF | 3.91 | 7.11 | 9.7 | 5.12 | 14.1 | 17.35 | 4.37 | 4.71 | 0.73 | 1.39 | 0.5 | 0.69 |
| 1 | 60 | Fz | STDDEV | 6.03 | 3.8 | 3.9 | 2.44 | 3.62 | 2.98 | 1.74 | 1.71 | 0.97 | 1.55 | 1.05 | 1.08 |
| 1 | 60 | Fz | MODFRQ | 1.47 | 5.29 | 9.84 | 13.4 | 17.59 | 24.27 | 39.73 | 32.24 | 0.54 | 0.39 | 0.3 | 0.56 |
| 1 | 60 | Cz | MEAN | 8.17 | 7.66 | 10.54 | 6.12 | 12.41 | 9.25 | 15.27 | 4.36 | 0.73 | 1.25 | 0.62 | 0.85 |
| 1 | 60 | Cz | MEANF | 3.13 | 6.61 | 11.29 | 5.88 | 15.19 | 16.5 | 4.47 | 4.28 | 0.59 | 1.12 | 0.44 | 0.74 |
| 1 | 60 | Cz | STDDEV | 2.85 | 3.1 | 5.55 | 2.55 | 4.12 | 2.45 | 1.67 | 1.41 | 0.56 | 1.22 | 0.75 | 1.35 |
| 1 | 60 | Cz | MODFRQ | 1.76 | 5.37 | 9.94 | 13.36 | 17.58 | 24.13 | 39.72 | 32.3 | 0.54 | 0.4 | 0.31 | 0.57 |
| 1 | 60 | Fz-Cz | COHE | 50.47 | 35.32 | 43.75 | 14.3 | 56.78 | 34.85 | 61.98 | 0 | 0.81 | 2.47 | 0.62 | 0.77 |
| 1 | 60 | Fz-Cz | PHASE | 16.32 | 15.97 | 12.42 | 14.5 | 9.03 | 12.55 | 0.15 | 7.67 | 1.29 | 1.1 | 1.77 | 1.38 |
| 1 | 60 | Fz/Cz | ASYM | 1.28 | 1.09 | 0.9 | 1.03 | 0.97 | 1.08 | 0.99 | 1.08 | 1.21 | 1.06 | 1.12 | 0.93 |
| 2 | 60 | F3 | MEAN | 7.87 | 7.22 | 7.28 | 5.94 | 12.26 | 14.21 | 12.75 | 5.77 | 0.99 | 1.22 | 0.59 | 0.59 |
| 2 | 60 | F3 | MEANF | 2.94 | 5.62 | 7.88 | 4.55 | 12.95 | 23.15 | 5.95 | 5.38 | 0.71 | 1.23 | 0.43 | 0.61 |
| 2 | 60 | F3 | STDDEV | 5.87 | 3.47 | 3.01 | 2.44 | 3.8 | 4.4 | 2.79 | 2.03 | 1.15 | 1.42 | 0.91 | 0.79 |
| 2 | 60 | F3 | MODFRQ | 1.5 | 5.29 | 10 | 13.42 | 17.7 | 24.22 | 39.89 | 32.42 | 0.53 | 0.39 | 0.3 | 0.56 |
| 2 | 60 | F4 | MEAN | 10.5 | 9.46 | 8.64 | 6.11 | 11.99 | 12.75 | 14.48 | 5.19 | 1.09 | 1.55 | 0.79 | 0.72 |
| 2 | 60 | F4 | MEANF | 3.75 | 7.48 | 9.71 | 4.8 | 12.67 | 19.73 | 4.52 | 5.14 | 0.77 | 1.56 | 0.59 | 0.77 |
| 2 | 60 | F4 | STDDEV | 6.73 | 4.37 | 3.65 | 2.4 | 4.1 | 3.82 | 1.98 | 1.92 | 1.2 | 1.82 | 1.06 | 0.89 |
| 2 | 60 | F4 | MODFRQ | 1.91 | 5.28 | 9.84 | 13.44 | 17.61 | 24.59 | 39.77 | 32.21 | 0.54 | 0.39 | 0.3 | 0.56 |
| 2 | 60 | F3-F4 | COHE | 47.78 | 38.45 | 39.2 | 12.58 | 47.35 | 35.68 | 36.12 | 0.53 | 0.98 | 3.06 | 0.81 | 0.83 |
| 2 | 60 | F3-F4 | PHASE | 16.87 | 16.07 | 14.52 | 21.13 | 18.2 | 26.65 | 4.18 | 29.12 | 1.11 | 0.76 | 0.88 | 0.8 |
| 2 | 60 | F3/F4 | ASYM | 0.75 | 0.76 | 0.84 | 0.97 | 1.02 | 1.12 | 0.88 | 1.11 | 0.91 | 0.78 | 0.75 | 0.82 |
| 3 | 60 | C3 | MEAN | 6.08 | 6.15 | 9.85 | 5.4 | 9.23 | 9.53 | 9.84 | 4.16 | 0.62 | 1.14 | 0.67 | 1.07 |
| 3 | 60 | C3 | MEANF | 2.94 | 5.8 | 11.8 | 6.07 | 13.79 | 20.22 | 4.72 | 5.13 | 0.49 | 0.96 | 0.42 | 0.86 |
| 3 | 60 | C3 | STDDEV | 2.98 | 2.61 | 4.66 | 2.1 | 3 | 3.4 | 1.9 | 1.45 | 0.56 | 1.25 | 0.87 | 1.55 |
| 3 | 60 | C3 | MODFRQ | 1.5 | 5.25 | 10.1 | 13.27 | 17.62 | 24.22 | 39.83 | 32.38 | 0.52 | 0.4 | 0.3 | 0.57 |
| 3 | 60 | C4 | MEAN | 6.7 | 6.85 | 9.24 | 5.43 | 9.63 | 7.98 | 12.59 | 3.35 | 0.74 | 1.26 | 0.71 | 0.96 |
| 3 | 60 | C4 | MEANF | 3.47 | 7.09 | 12.24 | 6.12 | 13.94 | 15.46 | 4.15 | 4.38 | 0.58 | 1.16 | 0.51 | 0.88 |
| 3 | 60 | C4 | STDDEV | 3.36 | 2.73 | 4.25 | 2.33 | 2.88 | 2.26 | 1.21 | 1.29 | 0.64 | 1.17 | 0.94 | 1.47 |
| 3 | 60 | C4 | MODFRQ | 1.86 | 5.27 | 9.94 | 13.3 | 17.51 | 24.38 | 39.65 | 32.19 | 0.53 | 0.4 | 0.3 | 0.57 |
| 3 | 60 | C3- | COHE | 35.65 | 21.57 | 33.58 | 9.18 | 33.57 | 19.85 | 7.23 | 0.12 | 0.64 | 2.35 | 0.64 | 1 |
| | | C4 | | | | | | | | | | | | | |
| 3 | 60 | C3- | PHASE | 12.38 | 14.93 | 27.25 | 26.25 | 18.77 | 28.63 | 3.15 | 22.28 | 0.55 | 0.57 | 0.8 | 1.45 |
| | | C4 | | | | | | | | | | | | | |
| 3 | 60 | C3/C4 | ASYM | 0.91 | 0.9 | 1.07 | 0.99 | 0.96 | 1.19 | 0.78 | 1.24 | 0.84 | 0.9 | 0.94 | 1.11 |
| 4 | 60 | P3 | MEAN | 5.55 | 5.55 | 11.81 | 5.52 | 7.39 | 7 | 7.49 | 3.07 | 0.47 | 1.01 | 0.75 | 1.6 |
| 4 | 60 | P3 | MEANF | 3.7 | 7.4 | 20.2 | 7.33 | 12.8 | 15.29 | 2.82 | 3.96 | 0.37 | 1.01 | 0.58 | 1.58 |
| 4 | 60 | P3 | STDDEV | 2.24 | 3.58 | 6.24 | 2.48 | 3.26 | 2.37 | 1.11 | 1.49 | 0.57 | 1.44 | 1.1 | 1.91 |
| 4 | 60 | P3 | MODFRQ | 1.54 | 5.31 | 10.07 | 13.2 | 17.46 | 24.15 | 39.62 | 32.26 | 0.53 | 0.4 | 0.3 | 0.58 |
| 4 | 60 | P4 | MEAN | 9 | 7.8 | 11.72 | 6 | 9.06 | 6.9 | 11.11 | 3.15 | 0.67 | 1.3 | 0.86 | 1.29 |
| 4 | 60 | P4 | MEANF | 4.12 | 8.12 | 16.58 | 6.47 | 13.08 | 13.32 | 3.43 | 3.37 | 0.49 | 1.26 | 0.62 | 1.27 |
| 4 | 60 | P4 | STDDEV | 2.9 | 4.71 | 6.13 | 2.56 | 3.38 | 1.91 | 1.03 | 1.13 | 0.77 | 1.84 | 1.39 | 1.81 |
| 4 | 60 | P4 | MODFRQ | 1.78 | 5.34 | 9.94 | 13.25 | 17.46 | 24.21 | 39.64 | 32.14 | 0.54 | 0.4 | 0.31 | 0.57 |
| 4 | 60 | P3-P4 | COHE | 40.35 | 25.08 | 47.15 | 10.08 | 26.97 | 12.42 | 0.63 | 0.03 | 0.53 | 2.49 | 0.93 | 1.75 |
| 4 | 60 | P3-P4 | PHASE | 22.27 | 16.2 | 22.23 | 24.77 | 17.8 | 21.95 | 1.43 | 17.77 | 0.73 | 0.65 | 0.91 | 1.25 |
| 4 | 60 | P3/P4 | ASYM | 0.62 | 0.71 | 1.01 | 0.92 | 0.82 | 1.01 | 0.67 | 0.97 | 0.71 | 0.77 | 0.87 | 1.23 |
| 5 | 60 | T3 | MEAN | 5.95 | 4.88 | 6.88 | 4.47 | 6.77 | 6.96 | 7.77 | 3.41 | 0.71 | 1.09 | 0.72 | 1.02 |
| 5 | 60 | T3 | MEANF | 3.35 | 5.88 | 12.24 | 6.43 | 13.5 | 18.99 | 4.4 | 5.18 | 0.48 | 0.91 | 0.44 | 0.91 |
| 5 | 60 | T3 | STDDEV | 3.24 | 1.99 | 3.18 | 2.07 | 2.75 | 2.61 | 1.21 | 1.34 | 0.62 | 0.96 | 0.72 | 1.16 |
| 5 | 60 | T3 | MODFRQ | 1.49 | 5.26 | 9.93 | 13.37 | 17.5 | 24.5 | 39.65 | 32.37 | 0.53 | 0.39 | 0.3 | 0.57 |
| 5 | 60 | T4 | MEAN | 8.2 | 7.01 | 8.56 | 5.05 | 8.92 | 5.91 | 12.22 | 3.05 | 0.82 | 1.39 | 0.79 | 0.96 |
| 5 | 60 | T4 | MEANF | 4.09 | 7.82 | 12.79 | 5.5 | 14.33 | 12.47 | 4.28 | 3.52 | 0.61 | 1.42 | 0.55 | 0.89 |
| 5 | 60 | T4 | STDDEV | 3.79 | 2.86 | 3.22 | 1.99 | 2.57 | 1.87 | 1.07 | 0.85 | 0.89 | 1.44 | 1.11 | 1.25 |
| 5 | 60 | T4 | MODFRQ | 1.77 | 5.24 | 9.87 | 13.32 | 17.56 | 24.28 | 39.6 | 32.36 | 0.53 | 0.39 | 0.3 | 0.56 |
| 5 | 60 | T3-T4 | COHE | 32.02 | 11.33 | 25.7 | 1.92 | 22.12 | 4.98 | 0.7 | 0.03 | 0.44 | 5.9 | 0.51 | 1.16 |
| 5 | 60 | T3-T4 | PHASE | 45.2 | 36.68 | 46.17 | 38.87 | 25.38 | 37.42 | 1.93 | 29.63 | 0.79 | 0.94 | 1.45 | 1.82 |
| 5 | 60 | T3/T4 | ASYM | 0.73 | 0.7 | 0.8 | 0.89 | 0.76 | 1.18 | 0.64 | 1.12 | 0.87 | 0.79 | 0.92 | 1.06 |
| 6 | 60 | O1 | MEAN | 5.12 | 4.39 | 7.08 | 4.26 | 4.09 | 5.14 | 1.98 | 2.68 | 0.62 | 1.03 | 1.07 | 1.73 |
| 6 | 60 | O1 | MEANF | 5.4 | 8.81 | 18.41 | 8.97 | 10.4 | 18.32 | 2.34 | 5.31 | 0.48 | 0.98 | 0.85 | 1.77 |
| 6 | 60 | O1 | STDDEV | 2.59 | 2.74 | 3.32 | 1.92 | 1.38 | 1.44 | 0.73 | 0.9 | 0.83 | 1.43 | 1.99 | 2.41 |
| 6 | 60 | O1 | MODFRQ | 1.57 | 5.24 | 10.02 | 13.24 | 17.32 | 24.65 | 39.85 | 32.27 | 0.52 | 0.4 | 0.3 | 0.58 |
| 6 | 60 | O2 | MEAN | 5.35 | 4.88 | 7.52 | 4.11 | 4.16 | 4.84 | 1.76 | 2.44 | 0.65 | 1.19 | 1.17 | 1.81 |
| 6 | 60 | O2 | MEANF | 5.9 | 9.79 | 20.45 | 8.75 | 10.34 | 16.86 | 2 | 4.46 | 0.48 | 1.12 | 0.95 | 1.98 |
| 6 | 60 | O2 | STDDEV | 2.57 | 3.01 | 3.39 | 1.64 | 1.54 | 1.65 | 0.65 | 0.74 | 0.89 | 1.83 | 1.95 | 2.2 |
| 6 | 60 | O2 | MODFRQ | 1.52 | 5.24 | 10.08 | 13.13 | 17.37 | 24.49 | 39.81 | 32.25 | 0.52 | 0.4 | 0.3 | 0.58 |
| 6 | 60 | O1- | COHE | 27.73 | 14 | 30.62 | 1.5 | 2.73 | 2.38 | 0 | 0.13 | 0.46 | 9.33 | 5.13 | 11.22 |
| | | O2 | | | | | | | | | | | | | |
| 6 | 60 | O1- | PHASE | 14.63 | 15.42 | 12.88 | 16.73 | 13.8 | 16.67 | 19.13 | 16.58 | 1.2 | 0.92 | 1.12 | 0.93 |
| | | O2 | | | | | | | | | | | | | |
| 6 | 60 | O1/O2 | ASYM | 0.96 | 0.9 | 0.94 | 1.04 | 0.98 | 1.06 | 1.12 | 1.1 | 0.96 | 0.87 | 0.91 | 0.96 |

It will be appreciated by persons skilled in the art that numerous variations and/or modifications may be made to the invention as described in the specific embodiments without departing from the spirit and scope of the invention as broadly described. The present embodiments are, therefore, to be considered in all respects as illustrative and not restrictive. Other features and aspects of this invention will be appreciated by those skilled in the art upon reading and comprehending this disclosure. Such features, aspects, and expected variations and modification of the reported results and examples are clearly within the scope of the invention where the invention is limited solely by the scope of the following claims.

The biofeedback system provides 8 positions, each selecting 4 channels. With a rear pushbutton, a 9^{th} position is available. The sensors for the positions are:

| Position | Active 1 | Active 2 | Active 3 | Active 4 |
|---|---|---|---|---|
| 1 | Fz | Cz | T3 | T4 |
| 2 | F3 | F4 | O1 | O2 |
| 3 | C3 | C4 | F7 | F8 |
| 4 | P3 | P4 | T5 | T6 |
| 5 | Fp1 | Fp2 | Pz | Oz (not 10/20) |
| 5a | T3 | T4 | Pz | Oz (not 10/20) |
| 6 | O1 | O2 | C3 | C4 |
| 7 | F7 | F8 | F3 | F4 |
| 8 | T5 | T6 | Fz | Cz |

In addition to taking EEG data for evaluation, the biofeedback system can also be used for training. In each position, a particular set of sites and connections is used. In each position, the biofeedback system provides 4 sites, and 6 connection paths between them. By using particular biofeedback system positions for training, it is possible to target specific brain functions in an efficient manner, and train all 4 sites.

When used with the Live Z-score training capability, it is possible to train all 4 sites, in addition to their 6 interconnections. This provides an efficient means to target specific functions.

When used with 4 channels, the live Z-score software provides 248 training variables as z scores: For each channel, for each of 8 bands: Absolute and relative power (4×16=64 z-scores). For each channel: 10 power ratios (4×10=40 z-scores). For each pair of channels (6 pairs) coherence, phase, asymmetry (6×24=144 z-scores)

The following pages detail the brain locations and functions accessed by each biofeedback system position, based upon the cited paper by Walker et al (2007). Each position provides a “window” into the trainee's brain, with unique capabilities for assessment and training. By referring to these charts, along with the live z-scores, it becomes possible to monitor and train specific brain functions using 4 channels in a convenient and optimal manner.

Based upon the following detailed explanations, each of the 9 possible biofeedback system settings becomes a “window” into particular aspects of brain function. When the brain is analyzed by taking sets of 4 channels in particular patterns, each pattern demonstrates a particular set of brain functional elements, and their interactions.

For purposes of general understanding, it is possible to classify each biofeedback system position in terms of the brain activities that it reflects, and how these are integrated into the overall function of the brain. In addition, by considering the effects of hypo- or hyper-coherence in each possible pair, it is possible to address modular interactions, and place them in the context of clinical signs. Each of the positions is described in detail on one of the following pages. For a summary account of their properties, the following nomenclature can emerge. For the benefit of succinctness, each position is further identified with an overall role, and a role “image” of that brain subsystem, the role that it subserves. It is anticipated that this interpretation will be of value in clinical assessment, and management of trainees, in cases in which particular functional subsystems can be identified for purposes of optimizing clinical outcomes.

| Position | Brain Site(s) | Functional Aspects | Overall Role |
|---|---|---|---|
| 1 | Frontal; Temporal | Remembering and | Goalsetting; |
| | | Planning | “Captain” |
| 2 | Frontal; Occipital | Seeing and Planning | Lookout; “Guide” |
| 3 | Central; Frontal | Doing and Expressing | Outward |
| | | | Expression; |
| | | | “Actor’ |
| 4 | Parietal; Temporal | Perceiving and | Interpreting the |
| | | Understanding | world; “Scholar” |
| 5 | Prefrontal; Parietal | Attending and | Observer; “Owl” |
| | | Perceiving | |
| 5a | Temporal; Parietal | Remembering and | Ponderer; “Sage” |
| | | Perceiving | |
| 6 | Occipital; Central | Seeing and Acting | Outward Actions; |
| | | | “Hero” |
| 7 | Frontal | Planning and | Planner, “Oracle” |
| | | Expressing | |
| 8 | Temporal; | Understanding and | Skilled; “Adept” |
| | Frontocentral | Doing | |

It is evident based upon this arrangement that this method provides a useful way to separate out functional subsystems in the brain, and to assess and train them in a systematic manner, using 4 channels of EEG. Depending on the outcome of the entire biofeedback system analysis, it becomes possible to define the functional aspects that are addressed by each of the possible biofeedback system positions, and to design training protocols around them.

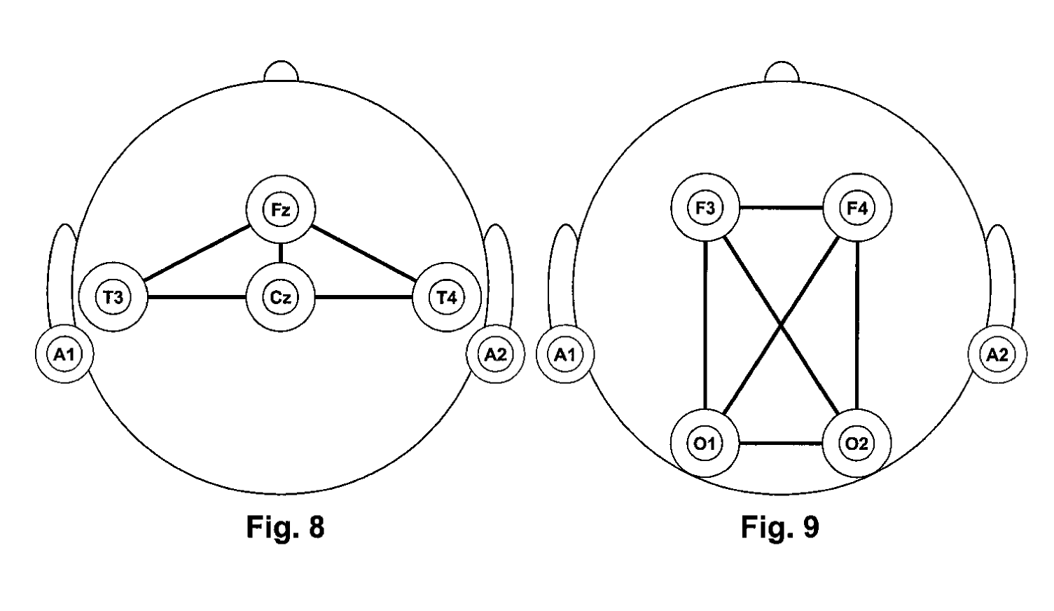

As shown in FIG. 8, position 1 uses electrode sensors Fz, Czr, T3, and T4, the frontal midline and temporal lobe sites. This position provides a primary window to motor planning of the lower extremities, sensorimotor integration, and logical and emotional memory formation and storage. Secondary functions include phonological processing, hearing, and ambulation.

| 10/20 Territory | | |
|---|---|---|
| Modules | Principal Function | Other Functions |
| Fz | Motor planning of both lower | Running, walking, |
| | extremities (BLE) and midline | kicking |
| Cz | Sensorimotor integration both | Ambulation |
| | lower extremities (BLE) and | |
| | midline | |
| T3 | Logical (verbal) memory | Phonological processing, |
| | formation and storage | hearing (bilateral), |
| | | suppression of tinnitus |
| T4 | Emotional (non-verbal) | Hearing (bilateral), |
| | memory formation and | suppression of tinnitus, |
| | storage | autobiographical memory |
| | | storage |

| Coherence | Result of Hypocoherence | Result of Hypercoherence |
|---|---|---|
| Fz-Cz | Less efficient midline | Lack of flexibility of midline |
| | motor action/midline | motor action/midline |
| | sensorimotor integration | sensorimotor integration |
| Fz-T3 | Less efficient logical | Lack of flexibility of logical |
| | memory/midline motor | memory/midline motor actions |
| | actions | |
| Fz-T4 | Less efficient emotional | Lack of flexibility of emotional |
| | memory/midline motor | memory/midline motor actions |
| | actions | |
| Cz-T3 | Less efficient logical | Lack of flexibility of logical |
| | memory/midline | memory/midline sensorimotor |
| | sensorimotor integration | integration |
| Cz-T4 | Less efficient emotional | Lack of flexibility of emotional |
| | memory/midline | memory/midline sensorimotor |
| | sensorimotor integration | integration |
| T3-T4 | Less efficient logical | Lack of flexibility of logical |
| | memory/emotional memory | memory/emotional memory |

As shown in FIG. 9, position 2 uses electrode sensors F3, F4, O1 and O2, the frontal and occipital homologous sites. This position provides a primary window to motor planning of the upper extremities, motor actions, and visual processing. Secondary functions include fine motor coordination, mood elevation, pattern recognition, and visual sensations and perception.

| 10/20 Territory | | |
|---|---|---|
| Modules | Principal Function | Other Functions |
| F3 | Motor planning right upper | Fine motor coordination, |
| | extremity (RUE) | mood elevation |
| F4 | Motor planning left upper | Fine motor coordination |
| | extremity (LUE) | (left hand) |
| O1 | Visual processing | Pattern recognition, color |
| | right half of space | perception, movement |
| | | perception, black/white |
| | | perception, edge perception |
| O2 | Visual processing | Pattern recognition, color |
| | left half of space | perception, movement |
| | | perception, black/white |
| | | perception, edge perception |

| Coherence | Result of Hypocoherence | Result of Hypercoherence |
|---|---|---|
| F3-F4 | Less efficient motor actions | Lack of flexibility motor actions |
| | RUE/motor actions LUE | RUE/motor actions LUE |
| F3-O1 | Less efficient motor actions | Lack of flexibility of logical |
| | RUE/visual sensations R | memory/midline motor actions |
| F3-O2 | Less efficient motor actions | Lack of flexibility of emotional |
| | RUE/visual sensations L | memory/midline motor actions |
| F4-O1 | Less efficient motor actions | Lack of flexibility of motor |
| | LUE/visual sensations R | actions LUE/visual |
| | | sensations R |
| F4-O2 | Less efficient motor actions | Lack of flexibility of motor |
| | LUE/visual sensations L | actions LUE/visual |
| | | sensations L |
| O1-O2 | Less efficient visual | Lack of flexibility of visual |
| | sensations R/visual | sensations L/visual sensations R |
| | sensations L | |

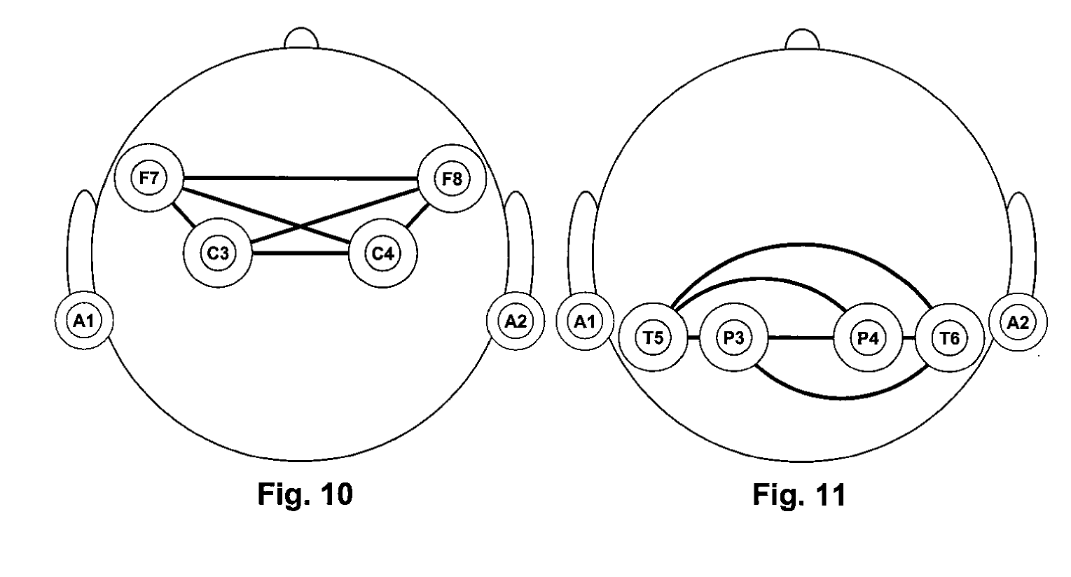

As shown in FIG. 10, position 3 uses electrode sensors C3, C4, F7 and F8, the mesial motor strip and lateral frontal homologous sites. This position provides a primary window to sensorimotor integration, and verbal and emotional expression, motor actions of the upper extremities, visual sensations, verbal/sensorimotor integration, and verbal/emotional expression. Secondary functions include alerting and calming responses, handwriting, drawing, and mood regulation.

| 10/20 Territory | | |
|---|---|---|
| Modules | Principal Function | Other Functions |
| C3 | Sensorimotor integration right | Alerting Responses |
| | upper extremity (RUE) | handwriting (right hand) |
| C4 | Sensorimotor integration left | Calming Handwriting |
| | upper extremity (LUE) | |
| F7 | Verbal Expression | Speech Fluency Mood |
| | | Regulation (cognitive) |
| F8 | Emotional Expression | Drawing (right hand) |
| | | Mood Regulation |
| | | (endogenous) |

| Coherence | Result of Hypocoherence | Result of Hypercoherence |
|---|---|---|
| C3-C4 | Less efficient sensorimotor | Lack of flexibility of sensorimotor |
| | integration RUE/sensorimotor | integration RUE/sensorimotor |
| | integration L | integration L |
| C3-F7 | Less efficient verbal sensorimotor | Lack of flexibility of |
| | integration RUE | verbal/sensorimotor integration RUE |
| C3-F8 | Less efficient emotional | Lack of flexibility of emotional |
| | expression/sensorimotor | expression/sensorimotor integration |
| | integration RUE | RUE |
| C4-F7 | Less efficient emotional | Lack of flexibility of emotional |
| | expression/sensorimotor | expression/sensorimotor integration LUE |
| | integration LUE | |
| C4-F8 | Less efficient emotional | Lack of flexibility of emotional |
| | expression/sensorimotor | expression/sensorimotor integration |
| | integration LUE | LUE |
| F7-F8 | Less efficient verbal/emotional | Lack of flexibility of |
| | expression | verbal/emotional expression |

As shown in FIG. 11, position 4 uses electrode sensors P3, P4, T5, and T6, the parietal and posterior temporal homologous sites. This position provides a primary window to perception and cognitive processing, spatial relations, and logical and emotional understanding, memory, and perceptions. Secondary functions include spatial relations sensations, calculations, multimodal interactions, and recognition of words and faces, and auditory processing.

| 10/20 Territory | | |
|---|---|---|
| Modules | Principal Function | Other Functions |
| P3 | Perception (cognitive | Spatial Relations, sensations, |
| | processing) right | multimodal sensations, |
| | half of space | calculations, praxis, reasoning |
| | | (verbal) |
| P4 | Perception (cognitive | Spatial relations, multimodal |
| | processing) left | interactions, praxis, reasoning |
| | half of space | (non-verbal) |
| T5 | Logical (verbal) | Word recognition, auditory |
| | understanding | processing |
| T6 | Emotional understanding | Facial recognition, symbol |
| | | recognition, auditory |
| | | processing |

| Coherence | Result of Hypocoherence | Result of Hypercoherence |
|---|---|---|
| P3-P4 | Less efficient perceptions | Lack of flexibility of perceptions |
| | R/perceptions L | R/perceptions L |
| P3-T5 | Less efficient logical | Lack of flexibility of logical |
| | memory/perception R | memory/perception R |
| P3-T6 | Less efficient emotional | Lack of flexibility of emotional |
| | memory/perceptions R | memory/perceptions R |
| P4-T5 | Less efficient logical | Lack of flexibility of logical |
| | memory/perceptions L | memory perception L |
| P4-T6 | Less efficient emotional | Lack of flexibility of emotional |
| | memory/perceptions L | memory/perceptions L |
| T5-T6 | Less efficient logical | Lack of flexibility of logical |
| | memory/emotional memory | memory/emotional memory |

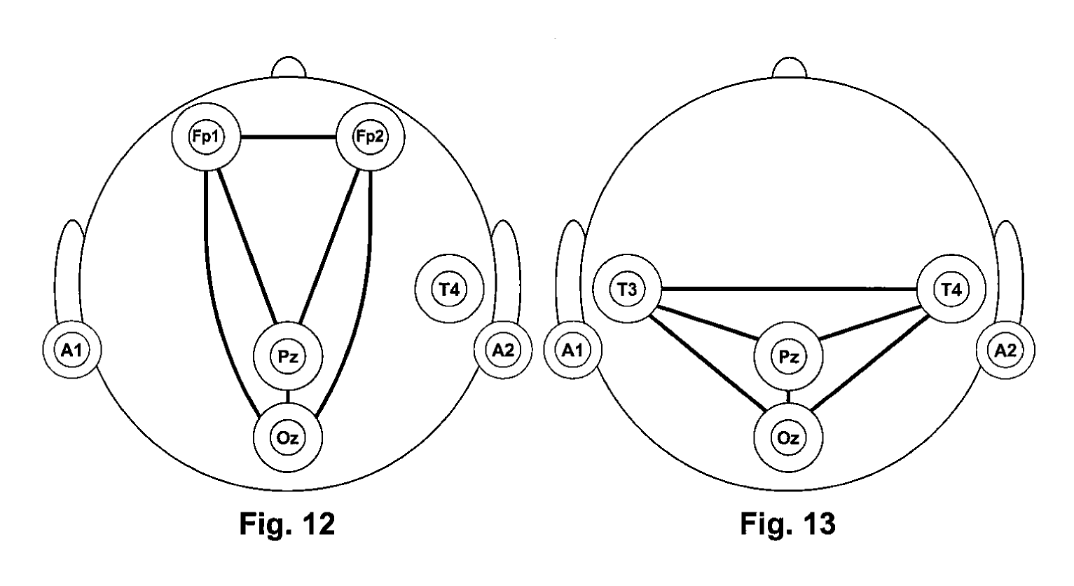

As shown in FIG. 12, position 5 uses electrode sensors Fp1, Fp2, Pz, and Oz, the prefrontal homologous and posterior midline sites. This position provides a primary window to logical and emotional attention, perception, and visual processing. Secondary functions include planning, decision making, task completion, sense of self, self-control, and route finding.

| 10/20 | | |
|---|---|---|
| Territory Modules | Principal Function | Other Functions |
| Fp1 | Logical Attention | Orchestrate network |
| | | interactions planning, decision |
| | | making, task completion, |
| | | working memory |
| Fp2 | Emotional Attention | Judgment, sense of self, self- |
| | | control, restraint of impulses |
| Pz | Perception midline | Spatial relations, praxis, route |
| | | finding |
| Oz (not a 10-20 | Visual processing | Primary visual sensation |
| position) | of space | |

| Coherence | Result of Hypocoherence | Result of Hypercoherence |
|---|---|---|
| Fp1-Fp2 | Less efficient integration of | Lack of flexibility of integrating |
| | logical/emotional attention | logical/emotional attention |
| Fp1-Pz | Logical attention/midline | Lack of flexibility of logical |
| | perception | attention/midline perception |
| Fp1-Oz | (no data) | (no data) |
| Fp2-Pz | Less efficient emotional | Lack of flexibility of emotional |
| | attention/midline perception | attention/midline perception |
| Fp2-Oz | (no data) | (no data) |
| Pz-Oz | (no data) | (no data) |

As shown in FIG. 13, position 5a uses electrode sensors T3, T4, Pz, and Oz, the temporal lobes and posterior midline sites. This position provides a primary window to logical and emotional attention, perception, and visual processing. Secondary functions include planning, decision making, task completion, sense of self, self-control, and route finding.

| 10/20 | | |
|---|---|---|
| Territory Modules | Principal Function | Other Functions |
| T3 | Logical (verbal) memory | Phonological processing, |
| | formation and storage | hearing (bilateral), |
| | | suppression of tinnitus |
| T4 | Emotional (non-verbal) | Hearing (bilateral), |
| | memory formation and | suppression of tinnitus, |
| | storage | autobiographical memory, |
| | | storage |
| Pz | Perception midline | Spatial relations, praxis, |
| | | route finding |
| Oz (not a 10-20 | Visual processing of | Primary visual sensation |
| position) | space | |

| Coherence | Result of Hypocoherence | Result of Hypercoherence |
|---|---|---|
| T3-T4 | Less efficient logical | Lack of flexibility of logical |
| | memory/emotional memory | memory/emotional memory |
| T3-Pz | Less efficient logical | Lack of flexibility of logical |
| | memory/midline perception | memory/midline perception |
| T3-Oz | (no data) | (no data) |
| T4-Pz | Less efficient logical | Lack of flexibility of logical |
| | memory/midline perception | memory/midline perception |
| T4-Oz | (no data0 | (no data) |
| Pz-Oz | (no data) | (no data) |

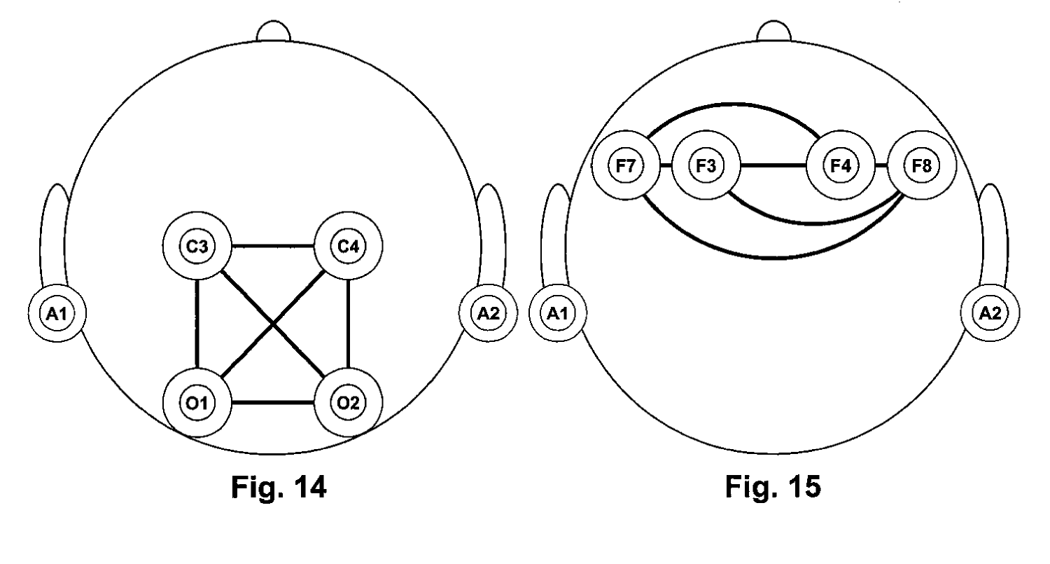

As shown in FIG. 14, position 6 uses electrode sensors O1, O2, C3, and C4, the occipital and motor strip homologous sites. This position provides a primary window to visual sensory processing, and sensorimotor integration of the upper extremities. Secondary functions include pattern recognition, perception of color, movement, black/white, and edges, alerting and calming responses, handwriting, and logical and emotional memory and perception.

| 10/20 Territory | | |
|---|---|---|
| Modules | Principal Function | Other Functions |
| O1 | Visual processing right half | Pattern recognition, color |
| | of space | perception, movement |
| | | perception, black/white |
| | | perception, edge perception |
| O2 | Visual processing left half | Pattern recognition, color |
| | of space | perception, movement |
| | | perception, black/white |
| | | perception, edge perception |
| C3 | Sensorimotor integration | Alerting responses, |
| | right upper extremity | handwriting (left hand) |
| | (RUE) | |
| C4 | Sensorimotor integration | Calming, handwriting (left |
| | left upper extremity | hand) |

| Coherence | Result of Hypocoherence | Result of Hypercoherence |
|---|---|---|
| O1-O2 | Less efficient visual sensations | Lack of flexibility of visual |
| | R/visual sensations L | sensations L/visual sensations R |
| O1-C3 | Less efficient sensorimotor | Lack of flexibility of sensorimotor |
| | integration RUE/visual sensations R | integration RUE/visual sensations R |
| O1-C4 | Less efficient sensorimotor | Lack of flexibility of sensorimotor |
| | integration LUE/visual sensations | integration LUE/visual sensations |
| O2-C3 | Less efficient sensorimotor | Lack of flexibility of sensorimotor |
| | integration RUE/visual sensations L | integration RUE/visual sensations L |
| O2-C4 | Less efficient sensorimotor | Lack of flexibility of sensorimotor |
| | integration LUE/visual sensations | integration LUE/visual sensations |
| C3-C4 | Less efficient sensorimotor | Lack of flexibility of sensorimotor |
| | integration RUE/sensorimotor | integration RUE/sensorimotor |
| | integration L | integration L |

As shown in FIG. 15, position 7 uses electrode sensors F7, F8, F3, and F4, the full frontal lobes homologous sites. This position provides a primary window to verbal and emotional expression, motor planning of the upper extremities, and motor actions. Secondary functions include speech fluency, mood regulation, and fine motor coordination.

| 10/20 Territory | | |
|---|---|---|
| Modules | Principal Function | Other Functions |
| F7 | Verbal expression | Speech fluency, mood |
| | | regulation (cognitive) |
| F8 | Emotional expression | Drawing (right hand), mood |
| | | regulation (endogenous) |
| F3 | Motor planning right upper | Fine motor coordination, |
| | extremity (RUE) | mood elevation |
| F4 | Motor planning left upper | Fine motor coordination |
| | extremity (LUE) | (left hand) |

| Coherence | Result of Hypocoherence | Result of Hypercoherence |
|---|---|---|
| F7-F8 | Less efficient verbal/ | Lack of flexibility of |
| | emotional expression | verbal/emotional expression |
| F7-F3 | Less efficient verbal/motor | Lack of flexibility of verbal/ |
| | actions R | motor actions R |
| F7-F4 | Less efficient verbal/motor | Lack of flexibility of verbal/ |
| | actions L | motor actions RUE |
| F8-F3 | Less emotional expression/ | Lack of flexibility of emotional |
| | motor actions RUE | expression/motor actions RUE |
| F8-F4 | Less emotional expression/ | Lack of flexibility of emotional |
| | motor actions LUE | expression/motor actions LUE |
| F3-F4 | Less efficient motor actions | Lack of flexibility motor actions |
| | RUE/motor actions LUE | RUE/motor actions LUE |

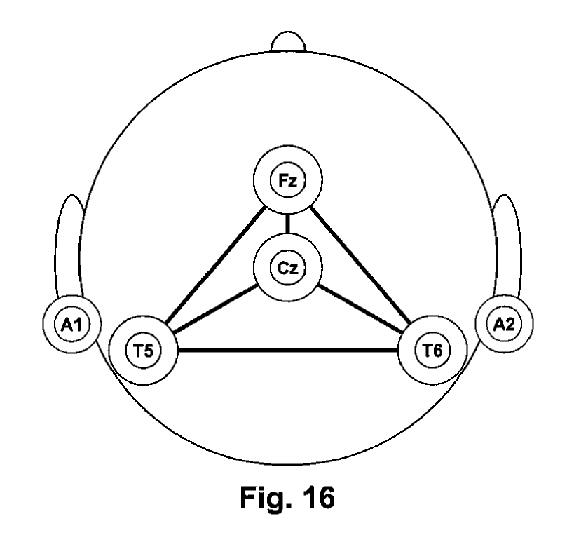

As shown in FIG. 16, position 8 uses electrode sensors T5, T6, Fz, and Cz, the posterior temporal and frontal midline sites. This position provides a primary window to logical and emotional understanding and memory, motor planning of the lower extremities, and sensorimotor integration. Secondary functions include word recognition, auditory processing, recognition of faces and symbols, running, walking kicking, and ambulation.

| 10/20 Territory | | |
|---|---|---|
| Modules | Principal Function | Other Functions |
| T5 | Logical (verbal) understanding | Word recognition, |
| | | auditory processing |
| T6 | Emotional understanding | Facial recognition, |
| | | symbol recognition, |
| | | auditory processing |
| Fz | Motor planning of both lower | Running, walking, |
| | extremities (BLE) and midline | kicking |
| Cz | Sensorimotor integration both | Ambulation |
| | lower extremities (BLE) and | |
| | midline | |

| Coherence | Result of Hypocoherence | Result of Hypercoherence |
|---|---|---|
| T5-T6 | Less efficient logical | Lack of flexibility of logical |
| | memory/emotional memory | memory/emotional memory |
| T5-Fz | Less efficient logical | Lack of flexibility of logical |
| | memory/midline motor | memory/midline motor actions |
| | actions | |
| T5-Cz | Less efficient logical | Lack of flexibility of logical |
| | memory/midline | memory/midline sensorimotor |
| | sensorimotor integration | integration |
| T6-Fz | Less efficient emotional | Lack of flexibility of emotional |
| | memory/midline motor | memory/midline motor actions |
| | actions | |
| T6-Cz | Less efficient emotional | Lack of flexibility of emotional |
| | memory/midline | memory/midline sensorimotor |
| | sensorimotor integration | integration |
| Fz-Cz | Less efficient midline | Lack of flexibility of midline |
| | motor action/midline | motor action/midline |
| | sensorimotor integration | sensorimotor integration |

What is claimed is:

1. A biofeedback system for administration of electroencephalographic (EEG) neurofeedback training, the system comprising:
a plurality of electrode sensors for placement on the head of a trainee;
a switching head box comprising a plurality of contacts located at a plurality of contact positions, each of the plurality of contacts being connected to one of the plurality of electrode sensors;
an interface device connected to the switching head box, the interface device comprising at least two EEG signal amplifiers;
a computer comprising software for generating user-control functions which corresponds in real-time to EEG signals received by the interface device and processed by the computer; and wherein the switching head box comprises a switch comprising a first conductor at a first position which connects a first electrode sensor to a first EEG signal amplifier of the interface device, and a second conductor at a second position which connects a second electrode sensor to a second EEG signal amplifier, for transmitting EEG signals from the trainee to the computer, the system comprising a combination of the electrode sensors of an EEG guided biofeedback system for providing EEC guided biofeedback and neural training on:
motor planning of the lower extremities and midline; sensorimotor integration of both lower extremities and midline; logical (verbal) memory formation and storage; emotional (non-verbal) memory formation and storage; motor planning right upper extremity; motor planning left upper extremity; right half of space; left half of space; frontal and occipital homologous sites of the brain or motor planning of the upper extremities, motor actions or visual processing.
electrode sensor Fz, whose principal function is the motor planning of the lower extremities and midline;
electrode sensor Cz, whose principal function is the sensorimotor integration of both lower extremities and midline;
electrode sensor T3, whose principal function is logical (verbal) memory formation and storage;
electrode sensor T4, whose principal function is emotional (non-verbal) memory formation and storage;
wherein the biofeedback system contains at least a four channel, 5-position switching head box;
wherein this combination of electrode sensors focuses on the frontal midline and temporal lobes; and
wherein this combination of electrode sensors provides neural feedback relating to motor planning of the lower extremities; sensorimotor integration; and logical and emotional memory formation and storage.

2. The biofeedback system of claim 1 comprising a combination of electrode sensors consisting of:
electrode sensor F3, whose principal function is motor planning right upper extremity;
electrode sensor F4, whose principal function is motor planning left upper extremity;
electrode sensor O1, whose principal function is visual processing right half of space;
electrode sensor O2, whose principal function is visual processing left half of space;
wherein the biofeedback system contains at least a four channel, 5-position switching head box;
wherein this combination of electrode sensors focuses on the frontal and occipital homologous sites of the brain; and
wherein this combination of electrode sensors provides neural feedback relating to motor planning of the upper extremities; motor actions; and visual processing.

3. The biofeedback system of claim 1 consisting of:
electrode sensor C3, whose principal function is sensorimotor integration right upper extremity;
electrode sensor C4, whose principal function is sensorimotor integration left upper extremity;
electrode sensor F7, whose principal function is verbal expression;
electrode sensor F8, whose principal function is emotional expression;
wherein the biofeedback system contains at least a four channel, 5-position switching head box;
wherein this combination of electrode sensors focuses on the mesial motor strip and lateral frontal homologous sites of the brain; and
wherein this combination of electrode sensors provides neural feedback relating to sensorimotor integration, verbal and emotional expression, motor actions of the upper extremities, visual sensations, verbal/sensorimotor integration, and verbal/emotional expression.

4. The biofeedback system of claim 1 consisting of:
electrode sensor P3, whose principal function is perception (cognitive processing) right half of space;
electrode sensor P4, whose principal function is perception (cognitive processing) left half of space;
electrode sensor T5, whose principal function is logical (verbal) understanding;
electrode sensor T6, whose principal function is emotional understanding;
wherein the biofeedback system contains at least a four channel, 5-position switching head box;
wherein this combination of electrode sensors focuses on the parietal and posterior temporal homologous sites of the brain; and
wherein this combination of electrode sensors provides neural feedback relating to perception and cognitive processing, spatial relations, and logical and emotional understanding, memory, and perceptions.

5. The biofeedback system of claim 1 consisting of:
electrode sensor Fp1, whose principal function is logical attention;
electrode sensor Fp2, whose principal function is emotional attention;
electrode sensor Pz, whose principal function is perception midline;
electrode sensor Oz, whose principal function is visual processing of space;
wherein the biofeedback system contains at least a four channel, 5-position switching head box;
wherein this combination of electrode sensors focuses on the prefrontal homologous, and posterior midline sites of the brain; and
wherein his combination of electrode sensors provides neural feedback relating to logical and emotional attention; perception; and visual processing.

6. The biofeedback system of claim 1 consisting of:
electrode sensor T3, whose principal function is logical (verbal) memory formation and storage;
electrode sensor T4, whose principal function is emotional (non-verbal) memory formation and storage;
electrode sensor Pz, whose principal function is perception midline;
electrode sensor Oz, whose principal function is visual processing of space;
wherein the biofeedback system contains at least a four channel, 5-position switching head box;
wherein this combination of electrode sensors focuses on the temporal lobes, and posterior midline; and
wherein, this combination of electrode sensors provides neural feedback relating to logical and emotional attention, perception, and visual processing.

7. The biofeedback system of claim 1 consisting of:
electrode sensor O1, whose primary function is visual processing right half of space;
electrode sensor O2, whose primary function is visual processing left half of space;
electrode sensor C3, whose primary function is sensorimotor integration right upper extremity;
electrode sensor C4, whose primary function is sensorimotor integration left upper extremity;
wherein the biofeedback system contains at least a four channel, 5-position switching head box;
wherein this combination of electrode sensors focuses on the occipital and motor strip homologous sites of the brain; and
wherein this combination of electrode sensors provides neural feedback relating to visual sensory processing, and sensorimotor integration of the upper extremities.

8. The biofeedback system of claim 1 consisting of:
electrode sensor F7, whose primary function is verbal expression;
electrode sensor F8, whose primary function is emotional expression;
electrode sensor F3, whose primary function is motor planning right upper extremity;
electrode sensor F4, whose primary function is motor planning left upper extremity;
wherein the biofeedback system contains at least a four channel, 5-position switching head box;
wherein this combination of electrode sensors focuses on the full frontal lobes homologous sites of the brain;
wherein this combination of electrode sensors provides neural feedback relating to verbal and emotional expression, motor planning of the upper extremities, and motor actions.

9. The biofeedback system of claim 1 consisting of:
electrode sensor T5, whose primary function is logical (verbal) understanding;
electrode sensor T6, whose primary function is emotional understanding;
electrode sensor Fz, whose primary function is motor planning of both lower extremities and midline;
electrode sensor Cz, whose primary function is sensorimotor integration, both lower extremities and midline;
wherein the biofeedback system contains at least a four channel, 5-position switching head box;
wherein this combination of electrode sensors focuses on the posterior temporal and frontal midline; and
wherein this combination of electrode sensors provides neural feedback relating to logical and emotional understanding and memory, motor planning of the lower extremities, and sensorimotor integration.
